(19) United States
(12) Patent Application Publication
(10) Pub. No.: US 2005/0180938 A1
(43) Pub. Date: Aug. 18, 2005

(54) Skin care cosmetic regime and kit

(76) Inventor: Beatrice Novelli, Geneva (CH)

Correspondence Address: Clifford W. Browning;Bank One Center/Tower, Suite 3700, 111 Monument Circle, Indianapolis, IN, 46204-5137, US

(21) Appl. No.: 10/780,090

(22) Filed: Feb. 17, 2004

(51) Int. Cl. A61K 7/06; A61K 7/11

(52) U.S. Cl. 424/70.13; 604/890.1

(57) ABSTRACT

Skin care cosmetic kit comprising a dermatological water vaporizer for projecting micronic water droplets on the surface of the skin to be treated, and a viscoelastic gel adapted for topical application on the skin surface after application of the water droplets, the viscoelastic gel comprising a hyaluronic acid salt having an average molecular weight greater than 500,000 Daltons at a concentration of 1.0 to 3.5% w/v in an aqueous solution.

The present invention relates to a skin care cosmetic regime and kit.

Among the numerous lotions and creams employed in the field of skin care for moistering, softening, restoring elasticity, and improving lubrication, certain are based on formulations comprising hyaluronic acid or salts thereof, in particular sodium hyaluronate, as described for example in U.S. Pat. No. 4,303,676.

Hyaluronic acid is a mucoid polysaccharide of biological origin, which is widely distributed in nature. For example, it is known that hyaluronic acid is present in various animal tissues such as umbilical cord, synovial fluid, vitreous humor, rooster comb and various connective tissues such as skin and cartilage.

Chemically, hyaluronic acid is a member of glycosaminoglycans and it is constituted by alternating and repeating units of D-glucuronic acid and N-acetyl-D-glucosamine, to form a linear chain having a molecular weight up to 13×10⁶ Daltons.

As a naturally occurring substance present in various human tissues, hyaluronic acid is non immunogenic. Moreover, in view of the important viscoelastic and hydrophilic properties of hyaluronic acid, it is known to be effective in skin hydration without significantly blocking the diffusion of normal skin metabolites. In a physiological solution, hyaluronic acid molecules form long coil structures occupying large domains even when present at fairly low concentrations that in fact provide “pores” to allow smaller molecules to pass through, in particular electrolytes and nutrients, while blocking the diffusion of larger molecules such as proteins. The binding of quite a large number of water molecules in the macromolecular structure of hyaluronic acid is a determining characteristic for the excellent moisturizing properties of hyaluronic acid formulations. Moreover, the viscoelastic properties of HA allow it to be effectively applied as a thin film on skin while retaining its moisturizing and lubricating properties.

Despite the incorporation of HA salts in certain known skin care cosmetic formulations, there is a desire to increase the effectiveness of skin care cosmetics, in particular to improve their moisturizing, softening, or lubricating properties, not only during or soon after topical application of the formulation, but days or even weeks thereafter. Conventional skin care cosmetic formulations are generally not effective in providing both immediate or rapid treatment of the skin, as well as a long lasting effect, while at the same time avoiding discomfort such as the presence of oily substances or visible films on the skin that are usually considered undesirable.

In view of the aforegoing, it is an object of this invention to provide a skin care cosmetic kit and a skin care cosmetic regime for effective and long lasting skin care.

It is advantageous to provide a skin care kit with components for topical application, and a skin care regime for application thereof, that creates a film that is discrete in terms of sensory or visual perception.

It is an advantage to provide a skin care cosmetic kit, and a skin care regime for application of the components thereof, that are particularly effective in the rapidity, effectiveness and duration of the treatment or restoration of skin properties, such as softness, elasticity, and moisture content.

It is advantageous to provide a skin care cosmetic kit, and a skin care regime, that provides an effective thermal barrier, particularly for skin types sensitive or allergic to cold environments.

Objects of this invention have been achieved by providing the skin care cosmetic kit according to claim 1, and a skin care cosmetic regime according to claim 11.

Disclosed herein is a skin care cosmetic kit comprising a dermatological water vaporizer for projecting micronic water droplets on the surface of the skin to be treated, and a viscoelastic gel adapted for topical application on the skin surface after application of the water droplets, the viscoelastic gel comprising a hyaluronic acid salt having an average molecular weight greater than 500,000 Daltons at a concentration of 1.5 to 3.5% w/v.

The hyaluronic acid salt is preferably sodium hyaluronate and the average molecular weight is preferably in the range of 1.5 to 2.5 million Daltons at a concentration preferably in the range of 1.5 to 2.5% w/v in an aqueous solution.

The water of the dermatological vaporizer is preferably a mineral water with a low mineral content, since experiments have shown that hard mineral waters, i.e. with high mineral content, somewhat reduce the binding effectiveness of the sodium hyaluronate of the gel in the skin surface.

The water droplets of the dermatological water vaporizer are preferably projected out of the vaporizer at a particle size less than 50 microns and preferably as small as approximately 5 microns.

In the skin care cosmetic regime according to this invention, the surface of skin to be treated is initially cleaned and dried, and subsequently sprayed with micronic water droplets from the dermatological water vaporizer. In this manner, water droplets can be applied very homogenously over the complex three-dimensional surface of the skin, in particular around the contours of eyes, in wrinkles and other irregularities of the skin surface. Subsequently, the high molecular weight HA gel is spread thinly and massaged over the surface of the wetted skin surface. The very fine and homogenous distribution of the water from the vaporizer enables the hydrophilic high molecular weight HA to bind effectively in a thin and uniform viscoelastic film over the irregular surface of the skin.

In this regard, the HA gel according to this invention, which preferably has a molecular weight in the range of 1.0 to 2.5 million Daltons and a fairly high concentration, preferably in the range of 1.9 to 2.1% w/v, has the advantageous property of capturing small water particles sprayed onto the skin by the vaporizer, thus enabling the HA to form a thin and uniform film that binds well to the surface of skin where it is applied. A very high concentration HA gel will tend to form a less uniform thin film in view of the increased hydrophilic nature which would tend to attract too much water into the HA micromolecular structure and thus somewhat diminish the binding of the HA to the skin surface. At the other extreme, an overly diluted gel, in particular with an HA concentration in aqueous solution of 1% w/v or less, does not bind so effectively to the skin surface previously wetted with the dermatological water vaporizer in view of the water strings or particles already captured in the macromolecular HA structure, diminishing somewhat the hydrophilic properties of the gel.

An important advantage of the skin care cosmetic regime according to this invention is that, by binding a thin and uniform film of high molecular weight HA on the skin surface, a film with excellent viscoelastic properties and water retention without blocking the diffusion of skin metabolites and nutrients, is thus provided. The latter properties also increase the residence time of the HA film on the skin, and thus the hydration and skin softening effectiveness. Moreover, the HA film thus produced is a particularly effective thermal barrier, and may be used as a formulation for reducing the effects such as skin reddening and other reactions of skin types sensitive or allergic to cold environments.

The cosmetic gel formulation may comprise, in addition to the HA in an aqueous solution, various preservative, bacteriostatic and fungistatic agents.

Specific examples of the formulations of the components comprised in the skin care cosmetic kit according to this invention are set forth hereafter.

Hydrogel Composition:

| Constituents | Quantity |
|---|---|
| Active ingredient | |
| Sodium hyaluronate (MW 1.5-2.5 10⁶ Da) | 1.90-2.10% w/v |
| Excipients | |
| Phenoxyethanol (bacteriostatic agent), | 0.80-0.90% w/v |
| Methylparaben (preservative), Ethylparaben | |
| (preservative), Butylparaben (fungistatic | |
| agent), Propylparaben (fungistatic agent), | |
| Isobutylparaben (preservative) | |
| Purified Water | q.s. ad |

Hydrogel Preferred Composition:

| Constituents | Quantity |
|---|---|
| Active ingredient | |
| Sodium hyaluronate (MW 1.8 10⁶ Da) | 2.00% w/v |
| Excipients | |
| Phenoxyethanol, Methylparaben, | 0.85 g/l |
| Ethylparaben, Butylparaben, | |
| Propylparaben, Isobutylparaben | |
| Purified Water | q.s. ad |

Preferably, the present formulations are in the form of neutral isotonic aqueous solutions with a pH ranging between 7.0 and 7.5, preferably around 7.3.

The concentration of sodium hyaluronate in the solution may range from 1.5% to 3.5%, and preferably is 2% by weight in respect of the volume of the solution

The hydrogel formulation has a viscosity ranging from 18 to 41 Pa.s (from 18,000 to 41,000 centipoises (cps)) at a shear rate of 2 sec-1 and at 25° C. The gel formulation of the present invention may be prepared according to known procedures, mixing the components under Good Manufacturing Practices Regulations (GMP) conditions by means of techniques and equipment usual in the preparation of compositions for topical use.

The dermatological water vaporizer may preferably be in the form of a compressed gas aerosol, adapted to project fine droplets of mineral water at a size of less than 50 microns, and preferably ≦5 microns.

The optimum propellant providing the driving force to expel the mineral water from the aerosol container is Nitrogen (N₂) under pressure.

The mineral water used in the dermatological water vaporizer according to this invention preferably comprises the following oligo-elements in the ranges specified (mg/l ):
Calcium (Ca): 0-90
Magnesium (Mg): 0-45
Sodium/Natrium (Na): 0-7
Potassium (K): 0-3
Bicarbonates HCO₃: 50-500
Sulphates SO₄: 5-15
Chloride Cl: 0-5
Nitrates: 0-5
Silicates: 0-15.

For example, the composition of a mineral water used in the trial of Table 1 is as follows:
Calcium (Ca): 78
Magnesium (Mg): 24
Sodium/Natrium (Na): 5
Potassium (K): 1
Bicarbonates HCO₃: 357
Sulphates SO₄: 10
Chloride Cl: 4,5
Nitrates: 3,8
Silicates: 13,5.
Ph: 7.2
At 180° dry residue: 309 ml/l
Propelling gas: nitrogen (N₂)

Heavily mineralized waters, such as the commercially available La Roche-Posay thermal source water, which has the following composition (mg/l)
Bicarbonates: 387
Calcium: 149
Silicates: 31.6
Magnesium (Mg): 4.4
Selenium: 53 μg
Copper, Zinc: ≦5 μg
Ph: 7
At 180° dry residue: 595 ml/l
Propelling gas: nitrogen (N₂) were found to give not entirely satisfactory results. In tests applying the cosmetic skin care regime with the aforementioned thermal source water on ten female subjects of different age, the subjects perceived a general irritation of the skin with pricking sensation, especially on sensitive skin areas, such as around the eyes.
   Skin Care Regime Trial:

A comparative study of the acceptability, harmlessness and performance of a facial skin care regime on ten female subjects using the dermatological water vaporizer plus different HA based gels was performed. The components used in the different trials and qualitative results attained are described in the tables hereafter.

The following regime was followed for five consecutive mornings, for each HA based gel of the trial: dermatological water vaporizer containing mineral water under pressure with neutral ph 7.2 as described above, was used to wet the upper layer of the skin of the face, nose and neck, followed by the topical administration and massage of the respective HA based gel formulation.

The female subjects were aged between 18 and 70 years old, and had one or more of the following complaints: Presence of skin dryness. Red skin sign following cold exposure (“red nose effect”). Patients who asked for a better hydration treatment of their skin.

The trial subjects evaluated their overall satisfaction based on the following scale:
0 unsatisfied.
1 not very satisfied.
2 satisfied.
3 very satisfied.

The results are summarized in the following tables, accompanied by a description of the composition of the formulation of the HA based gel used.

Trial Results:

Age of subjects: A=45 years
B=62 years
C=63 years
D=26 years
E=40 years
F=47 years
G=38 years
H=48 years
I=26 years

J=54 years

TRIAL 1: Dermatological Water Vaporizer +
Hydrogel according to the preferred composition

| | SUBJECTS | | | | | | | | | |
|---|---|---|---|---|---|---|---|---|---|---|
| CRITERIA | A | B | C | D | E | F | G | H | I | J |
| Hydration | 3 | 3 | 3 | 3 | 3 | 3 | 3 | 3 | 3 | 3 |
| sensation | | | | | | | | | | |
| Radiance | 2 | 3 | 2 | 2 | 3 | 2 | 2 | 3 | 2 | 3 |
| appearance | | | | | | | | | | |
| Skin softness | 3 | 3 | 3 | 2 | 3 | 3 | 2 | 3 | 3 | 3 |
| Thermic barrier | 3 | 3 | 3 | 3 | 3 | 3 | 3 | 3 | 3 | 3 |
| (“red nose effect”) | | | | | | | | | | |

TRIAL 2: Dermatological Water Vaporizer + HA 0.18% Composition

| | SUBJECTS | | | | | | | | | |
|---|---|---|---|---|---|---|---|---|---|---|
| CRITERIA | A | B | C | D | E | F | G | H | I | J |
| Hydration | 0 | 0 | 0 | 1 | 2 | 0 | 1 | 0 | 1 | 0 |
| sensation | | | | | | | | | | |
| Radiance | 1 | 2 | 1 | 0 | 1 | 1 | 1 | 1 | 2 | 1 |
| appearance | | | | | | | | | | |
| Skin softness | 1 | 1 | 0 | 1 | 0 | 0 | 1 | 1 | 1 | 1 |
| Thermic barrier | 0 | 1 | 1 | 1 | 1 | 0 | 1 | 1 | 1 | 0 |
| (“red nose effect”) | | | | | | | | | | |

HA 0,18% Composition:

The composition of HA 0,18% used in Trial 2 is as follows:

| | Quantity |
|---|---|
| Constituents | Amount (w/v) |
| Active ingredient | |
| Sodium hyaluronate (MW 0.5-0.8 10⁶ Da) | 180.0 mg/l |
| Excipients | |
| Sodium chloride | 279.2 mg/l |
| Potassium chloride | 103.3 mg/l |
| Sodium monohydrogen phosphate.12 H₂O | 322.2 mg/l |
| Sodium citrate | 26.0 mg/l |
| Magnesium chloride.6 H₂O | 9.2 mg/l |
| Calcium chloride.2 H₂O | 8.9 mg/l |
| Hydrochloric acid 10% q.s. ad | pH 7.2-7.4 |
| Purified Water q.s. ad | 100.0 ml |

HA 0.18% is a hypotonic solution (140-160 mOsm/l), adjusted to pH 7.2-7.4

Trial 3: Dermatological Water Vaporizer + HA 0.25% Composition

| | SUBJECTS | | | | | | | | | |
|---|---|---|---|---|---|---|---|---|---|---|
| CRITERIA | A | B | C | D | E | F | G | H | I | J |
| Hydration | 0 | 1 | 0 | 1 | 0 | 1 | 1 | 1 | 1 | 0 |
| sensation | | | | | | | | | | |
| Radiance | 2 | 1 | 2 | 2 | 1 | 1 | 1 | 1 | 2 | 1 |
| appearance | | | | | | | | | | |
| Skin softness | 2 | 2 | 1 | 1 | 1 | 1 | 1 | 1 | 2 | 1 |
| Thermic barrier | 0 | 0 | 0 | 0 | 1 | 1 | 2 | 1 | 1 | 0 |
| (“red nose effect”) | | | | | | | | | | |

HA 0,25% Composition:

The composition of HA 0,25% used in Trial 3 is as follows

| | Quantity |
|---|---|
| Constituents | Amount (w/v) |
| Active ingredient | |
| Sodium hyaluronate (MW 1.3-2.0 10⁶ Da) | 250.0 mg/l |
| Excipients | |
| Sodium chloride | 680.0 mg/l |
| Potassium chloride | 192.7 mg/l |
| Sodium monohydrogenphosphate.12H₂O | 322.1 mg/l |
| Sodium citrate.2H₂O | 30.8 mg/l |
| Magnesium chloride.6H₂O | 9.1 mg/l |
| Calcium chloride.2H₂O | 8.7 mg/l |
| Glucose monohydrate | 99.0 mg/l |
| Hydrochloric acid 10% q.s. ad | pH 7.2-7.4 |
| Purified Water q.s. ad | 100.0 ml |

TRIAL 4: Dermatological Water Vaporizer + HA 0.50% Composition

| | SUBJECTS | | | | | | | | | |
|---|---|---|---|---|---|---|---|---|---|---|
| CRITERIA | A | B | C | D | E | F | G | H | I | J |
| Hydration | 1 | 0 | 1 | 1 | 0 | 0 | 1 | 0 | 1 | 0 |
| sensation | | | | | | | | | | |
| Radiance | 1 | 1 | 0 | 1 | 2 | 1 | 1 | 1 | 1 | 1 |
| appearance | | | | | | | | | | |
| Skin softness | 1 | 1 | 1 | 1 | 1 | 0 | 1 | 1 | 2 | 1 |
| Thermic barrier | 0 | 0 | 0 | 0 | 0 | 1 | 1 | 0 | 2 | 1 |
| (“red nose effect”) | | | | | | | | | | |

HA 0,50% Composition:

The composition of HA 0,50% used in Trial 4 is as follows

| | Quantity |
|---|---|
| Constituents | Amount (w/v) |
| Active ingredient | |
| Sodium hyaluronate (MW 1.6-1.8 10⁶ Da) | 0.5 g/l |
| Excipients | |
| Sodium chloride | 0.88 g/l |
| Sodium monohydrogenphosphate.12H₂O | 0.05143 g/l |
| Sodium dihydrogenphosphate.2 H₂O | 0.005 g/l |
| Hydrochloric acid 10% q.s. ad | pH 7.3 |
| Purified Water q.s. ad | 100.0 ml |

TRIAL 5: Dermatological Water Vaporizer + HA 1.0% Composition

| | SUBJECTS | | | | | | | | | |
|---|---|---|---|---|---|---|---|---|---|---|
| CRITERIA | A | B | C | D | E | F | G | H | I | J |
| Hydration | 1 | 1 | 1 | 2 | 2 | 1 | 2 | 1 | 2 | 2 |
| sensation | | | | | | | | | | |
| Radiance | 1 | 2 | 1 | 2 | 1 | 1 | 2 | 1 | 1 | 2 |
| appearance | | | | | | | | | | |
| Skin softness | 1 | 2 | 2 | 1 | 2 | 2 | 2 | 2 | 2 | 1 |
| Thermic barrier | 1 | 2 | 1 | 2 | 1 | 1 | 1 | 2 | 1 | 1 |
| (“red nose effect”) | | | | | | | | | | |

HA 1,0% Composition:

The composition of HA 1,0% used in Trial 5 is as follows

| | Quantity |
|---|---|
| Constituents | Amount (w/v) |
| Active ingredient | |
| Sodium hyaluronate (MW 0.8-1.6 10⁶ Da) | 1.0 g/l |
| Excipients | |
| Sodium chloride | 0.85 g/l |
| Sodium monohydrogenphosphate.12 H₂O | 0.06 g/l |
| Sodium dihydrogenphosphate.2 H₂O | 0.005 g/l |
| Purified Water q.s.ad | 100 ml |

TRIAL 6: Dermatological Water Vaporizer + HA 1.5% Composition

| | SUBJECTS | | | | | | | | | |
|---|---|---|---|---|---|---|---|---|---|---|
| CRITERIA | A | B | C | D | E | F | G | H | I | J |
| Hydration | 2 | 1 | 1 | 2 | 2 | 2 | 1 | 1 | 2 | 1 |
| sensation | | | | | | | | | | |
| Radiance | 2 | 2 | 2 | 2 | 2 | 2 | 2 | 2 | 2 | 2 |
| appearance | | | | | | | | | | |
| Skin softness | 1 | 2 | 2 | 1 | 2 | 2 | 2 | 1 | 2 | 2 |
| Thermic barrier | 2 | 2 | 1 | 2 | 1 | 1 | 2 | 1 | 2 | 1 |
| (“red nose effect”) | | | | | | | | | | |

| | Quantity |
|---|---|
| Constituents | Amount (w/v) |
| Active ingredient | |
| Sodium hyaluronate (MW 1.0-2.0 10⁶ Da) | 1.50 g/l |
| Excipients | |
| Sodium chloride | 0.77 g/l |
| Sodium monohydrogenphosphate.12 H₂O | 0.06 g/l |
| Sodium dihydrogenphosphate.2 H₂O | 0.005 g/l |
| Purified Water q.s.ad | 100 ml |

HA 1,5% Composition:

The composition of HA 1,5% used in Trial 6 is as follows

All subjects tolerated the treatment well. The preferred gel composition (Trial 1) showed superior results compared to the compositions used in Trials 2-6, especially in the appreciation of Hydration Sensation and Thermic Barrier.

The above trials also demonstrate the improved cosmetic properties of HA based gels with high concentration (above 1% w/v), used in conjunction with the dermatological water vaporizer.

1. Skin care cosmetic kit comprising a dermatological water vaporizer for projecting micronic water droplets on the surface of the skin to be treated, and a viscoelastic gel formulation adapted for topical application on the skin surface after application of the water droplets, the viscoelastic gel formulation comprising a hyaluronic acid salt (HA) at a concentration of 1.0 to 3.5% w/v in an aqueous solution.

2. Skin care cosmetic kit according to claim 1 wherein the average molecular weight of the HA is greater than 500,000 Daltons.

3. Skin care cosmetic kit according to the preceding claim, wherein the viscoelastic gel formulation comprises a hyaluronic acid salt having an average molecular weight in the range of 1.0 to 2.5 million Daltons.

4. Skin care cosmetic kit according to claim 1, 2 or 3, wherein the viscoelastic gel formulation comprises hyaluronic acid salt at a concentration in the range of 1.5 to 2.5% w/v.

5. Skin care cosmetic kit according to the preceding claim, wherein the hyaluronic acid salt in the viscoelastic gel formulation has a concentration in the range of 1.9 to 2.1% w/v.

6. Skin care cosmetic kit according to claim 1, wherein the dermatological water vaporizer is adapted to project water droplets with an average diameter of less than 50 microns.

7. Skin care cosmetic kit according to the preceding claim, wherein the water droplets projected by the dermatological water vaporizer have an average size in the range of 5 microns or less.

8. Skin care cosmetic kit according to claim 1, wherein the water of the dermatological water vaporizer is a soft mineral water.

9. Skin care cosmetic kit according to the preceding claim, wherein the mineral water of the dermatological water vaporizer comprises oligo-elements in the ranges (mg/l):
Calcium (Ca): 0-90
Magnesium (Mg): 0-45
Sodium/Natrium (Na): 0-7
Potassium (K): 0-3
Bicarbonates HCO₃: 50-500
Sulphates SO₄: 5-15
Chloride Cl: 0-5
Nitrates: 0-5
Silicates: 0-15.

10. Skin care cosmetic kit according to claim 1 wherein the viscoelastic gel formulation further comprises preservative, bacteriostatic and fungistatic agents.

11. Skin care cosmetic regime comprising spraying micronic water droplets from said dermatological water vaporizer on clean and dry skin, and subsequently applying a thin film of said viscoelastic gel as set forth in claim 1.

12. Use of a skin care cosmetic kit according to claim 1 as a thermal barrier for reducing the sensitivity of skin to cold environments.

13. Use of a skin care cosmetic kit according to claim 1 as a moisturizer and skin softener.
